(19) United States
(12) Patent Application Publication
(10) Pub. No.: US 2011/0158911 A1
(43) Pub. Date: Jun. 30, 2011

(54) Methods for Screening for Antibiotic Compounds

(75) Inventors: Ambrose Cheung, Hanover, NH (US); Zhibiao Fu, King of Prussia, PA (US)

(73) Assignee: Trustees of Dartmouth College, Hanover, NH (US)

(21) Appl. No.: 12/968,943

(22) Filed: Dec. 15, 2010

Related U.S. Application Data
(63) Continuation-in-part of application No. PCT/US2010/040695, filed on Jul. 1, 2010.
(60) Provisional application No. 61/222,304, filed on Jul. 1, 2009.

(51) Int. Cl. A61K 49/00; C12Q 1/68; C07H 21/04; C12Q 1/18

(52) U.S. Cl. 424/9.2; 435/6.1; 536/23.1; 435/32

(57) ABSTRACT

The present invention is a method for screening drugs for antibiotic activity by screening a drug for activity to disrupt a toxin-antitoxin complex in a bacterial cell.

This application is a continuation-in-part of PCT/US2010/040695 filed Jul. 1, 2010, claims benefit of priority to U.S. Provisional Application Ser. No. 61/222,304, filed Jul. 1, 2009, the content of which is incorporated herein by reference in its entirety.

### BACKGROUND OF THE INVENTION

Toxin and antitoxin (TA) systems are commonly found in prokaryotes. These systems function to allow the organisms to rapidly adjust rates of protein and DNA synthesis in order to respond to external stimuli and/or stress (Gerdes et al. 2005. Nature Rev. Microbiol. 3:371-382). Under normal circumstances, TA genes are co-transcribed and co-translated as part of an operon so that both antitoxin and toxin are produced together within the cytosol to form an inert complex. Under specific stress, transcription of the TA promoter will be repressed, disrupting transcription and subsequently translation. As toxins are stable compounds while the antitoxins compounds are more labile and prone to proteolytic attack by bacterial Lon/ClpP proteases, disruption of transcription from the TA promoter will result in excess toxin and activity of the toxin in the cell. The target of such toxins may be mRNA, DNA gyrase or DNA helicase, where interaction of toxin with these targets leads to disruption of transcription and translation of genes responsible for important cellular processes.

Based on sequence homology and cellular targets, there are eight major TA systems that have been identified in prokaryotes (Gerdes et al. 2005. Nature Rev. Microbiol. 3:371-382; Kamphius et al. 2007. Protein Peptide Lett. 14:113-124). Among these TA systems is the MazEF system, which includes the toxin MazF and the antitoxin MazE. These two proteins form a linear heterohexamer made up of alternating toxin and antitoxin homodimers (Kamada et al. 2003. Mol. Cell. 11:875-884). The MazEF TA complex in E. coli has been shown to autoregulate by binding of the DNA by the N-terminal domain of MazE. The MazF toxin has been shown to cleave translated mRNAs and through this mechanism to block protein synthesis within prokaryotic cells (Christensen et al. 2003. J. Mol. Biol. 332:809-819). The cleavage of mRNAs in E. coli is at ACA sites (Zhang et al. 2003. Mol. Cell. 12:913-923). A variety of conditions have been shown to trigger the activity of MazF in prokaryotic cells including, for example, stress linked to high temperatures, oxidative stress, DNA damage by thymidine starvation, UV irradiation, and contact with protein-inhibiting antibiotics (Kamphius et al. 2007. Protein Peptide Lett. 14:113-124). Although MazEF clearly functions as a bacteriostatic system (Gerdes et al. 2005. Nature Rev. Microbiol. 3:371-382) within prokaryotic cells, it is not clear whether MazEF also functions within cells as a system for programmed cell death.

Sequence analysis has revealed that the MazF toxin is more conserved among different bacteria than is the antitoxin MazE. This finding is consistent with the finding that the activity of similar TA systems in different bacteria is dependent on the specificity of the antitoxin. In fact, it has been found that Staphylococcus aureus MazEF homologs are quite different from E. coli MazEF homologs (Fu et al. 2007. J. Bacteriol. 189:8871-8879; Fu et al. 2009. J. Bacteriol. 191:2051-2059; Niles et al. 2009. J. Bacteriol. 191:2795-2805). It has been found through transcriptional analysis that the mRNA target of the toxin MazF in S. aureus is selective, sparing important transcripts such as gyrA and recA (Fu et al. 2009. J. Bacteriol. 191:2051-2059). Therefore, in S. aureus MazF has features of a bacteriostatic effect rather than a bacteriocidal effect. The toxic effect of MazF can be rescued by the antitoxin within a fixed time window. However, the effect of MazF, if prolonged, can lead to nonviable cells.

Based on the importance of TA systems within cells, including the MazEF system, interest has grown in the use of these systems in the development of new antibiotic compounds. To date, the only organisms not identified as having MazEF systems are Mycobacerium leprae, Chlymidia, Rickettsia, and Mycoplasm. MazEF has been found to be an important TA system within a variety of prokaryotes including E. coli, S. aureus, and S. pneumonia. There remains a need for methods to identify new antibiotic compounds active against clinically important bacteria.

### SUMMARY OF THE INVENTION

The present invention is a method of screening a drug for activity to disrupt a toxin-antitoxin complex in a bacterial cell by contacting a TA complex with a test drug and determining whether the test drug increases the amount of cleavage of RNA by the toxin, wherein an increase in the amount of cleavage of RNA by the toxin is indicative of activity to disrupt a toxin-antitoxin complex in the bacterial cell. In some embodiments, the TA complex is a MazEF complex, e.g., from a S. aureus cell. In other embodiments, cleavage of the RNA by the toxin is determined by measuring cleavage of a synthetic RNA substrate, e.g., containing a MazF recognition site (AUUC), wherein said RNA is flanked on each end by at least four deoxyribonucleotides. In further embodiments, the synthetic RNA substrate includes a fluorescent marker-quencher pair. In particular embodiments, cleavage of RNA by the toxin of a TA complex is determined by measuring cleavage of a synthetic RNA-DNA substrate comprising the 4-base MazF recognition site (AUUC) flanked on each side by at least four deoxyribonucleotides, wherein said substrate comprises a fluorescence marker at one end and a quencher at the other end. A synthetic substrate of use in the instant method is also provided.

The present invention is a method of screening a drug for activity to disrupt a toxin-antitoxin complex in a whole cell bacterial cell assay which comprises contacting a bacterial cell expressing a wild-type toxin-antitoxin complex with a test drug, contacting a bacterial cell that does not express the toxin-antitoxin complex with the test drug, and determining whether the test drug reduces the growth of the bacterial cell expressing the wild-type toxin-antitoxin complex as compared to the growth of the bacterial cell that does not express the toxin-antitoxin complex, wherein a reduction in bacterial growth is indicative of activity to disrupt a toxin-antitoxin complex in the wild-type bacterial cell. In preferred embodiments the bacterial strain is selected from the group consisting of S. aureus, E. coli 0157. Listeria, and M. tuberculosis, and the bacterial strains tested are wild-type S. aureus versus MazEFsa mutant S. aureus.

The present invention is a method of screening a drug for efficacy as an antibiotic which comprises inoculating a first mouse with a bacterial strain expressing a wild-type toxin-antitoxin complex, inoculating a second mouse with a bacterial strain a bacterial strain that does not express the wild-type toxin-antitoxin complex, administering to both the first mouse and the second mouse a test drug and measuring a volume of bacterial abscess that forms subcutaneously on said first mouse and said second mouse, wherein a reduction in said bacterial abscess volume in the first mouse when compared with abscess volume in the second mouse is indicative of efficacy of the test drug as an antibiotic compound. In preferred embodiments the bacterial strain is selected from the group consisting of S. aureus, E. coli 0157. Listeria, and M. tuberculosis, and the bacterial strains tested are wild-type S. aureus versus MazEFsa mutant S. aureus.

### DETAILED DESCRIPTION OF THE INVENTION

The present invention is a screening assay for identifying compounds that disrupt a TA complex within a prokaryotic organism. Disruption of the TA complex within a bacterial cell leads to either cell death or a bacteriostatic effect, depending on the activity of the TA system within the cell. The MazEF complex is found in a variety of bacteria and as such has now been found to be a useful target TA system for developing a screening assay. It has been found that the MazEF complex within S. aureus is a particularly useful target system. The screening assay is either a whole cell assay or a substrate assay. Also provided by the present invention is an in vivo efficacy assay for identifying compounds that have antibiotic activity and thus useful for treating a bacterial infection. In the context of the present invention efficacy is defined as the ability of a drug to produce a statistically significant decrease in bacterial cell growth. The in vivo assay is based on testing drugs for efficacy in a mouse model of subcutaneous infection wherein the model involves a mouse infected with either a wild-type or mutant bacterial strain and comparing the growth of the bacteria in animals infected with the wild-type versus the mutant bacterial strain. Compounds that disrupt the toxin-antitoxin complex of the wild-type strain will show reduced bacterial growth as compared to growth in animals with a strain that does not express the wild-type toxin-antitoxin complex.

The screening methods of the present invention were developed based on experiments to identify the cleavage site on mRNA that is cleaved by MazF in S. aureus. In the first experiments, the cleavage site of the MazF toxin of S. aureus on mRNA was found to be VUUV′ where V and V′ are adenine (A), cytosine (C) or guanine (G), but not uracil (U). As RNA is unstable and prone to RNase degradation from the environment, it would be preferable to use an RNA-hybrid template to screen for compounds that disrupt the MazEF complex in order to detect toxin activity. Therefore, a hybrid RNA-DNA hybrid molecule, which is less amenable to RNase-mediated cleavage, was developed. The hybrid molecule was synthesized that contained 12 bases. The hybrid contained the 4-base recognition site (AUUC) of MazF from S. aureus, and flanked the four bases on each side with four DNA bases to protect the hybrid from RNase-mediated cleavage. On one end of the hybrid molecule, the fluorescence marker FAM-6 was attached while at the other end the quencher BHG-1 was attached. The resulting RNA-DNA hybrid molecule did not yield significant fluorescence unless the RNA target site was cleaved by the MazF toxin to separate FAM-6 from BHG-1. Moreover, exposure of the 12-base hybrid molecule to the MazF toxin yielded fluorescence comparable to and even at levels lower than RNase A, which has been shown to efficiently cleave single-stranded RNA. This hybrid molecule was then used as the substrate for development of an antibiotic screening assay.

Thus, a screening assay was developed wherein MazEF complex of S. aureus was incubated with the hybrid molecule substrate and a compound to be tested for activity to disrupt the MazEF complex. In accordance with this assay, compounds that disrupt the complex will cleave the hybrid fluorescence substrate and yield detectable fluorescence. In one embodiment, multi-well plates are loaded with microliter amounts of the hybrid fluorescent substrate, the test compound and the MazEF complex. Control samples contain no MazEF complex. Results have been collected with S. aureus and the assay has been validated for use with S. aureus and activity of the MazEF complex.

As one of skill will appreciate, this screening assay can be modified to accommodate detection of disruption of the TA complex from any organism as long as the cleavage site of the toxin on RNA is identified.

Thus, the present invention is a method of screening drugs for activity to disrupt a toxin-antitoxin complex in a bacterial cell by contacting a TA complex with a test drug and determining whether the test drug increases the amount of cleavage of RNA by the toxin, wherein an increase in the amount of cleavage of RNA by the toxin is indicative of activity to disrupt a toxin-antitoxin complex in the bacterial cell. As described herein, there are a number of well-known toxin-antitoxin complexes that can be used in the assay of this invention. These include, but are not limited to, the MazEF module of, e.g., E. coli, S. aureus, S. pneumonia or M. tuberculosis; the relBE module of M. tuberculosis; and the higBA and yoeb/yefm modules. See Buts et al. 2005. Trends Biochem. Sci. 30:672-9; Gerdes et al. 2005. Nature Rev. Microbiol. 3:371-382; and Condon 2006. Mol. Microbiol. 61:573-583. In one embodiment, the toxin-antitoxin complex is a MazEF complex. In a particular embodiment the toxin-antitoxin complex is a MazEF complex from S. aureus.

As demonstrated herein, release of toxin from the TA complex allows for cleavage of its RNA substrate, an activity which can be detected using fluorescent substrates. In this respect, certain embodiments of this invention include the use of a synthetic RNA fluorescent substrate. In a more preferred embodiment, the synthetic RNA fluorescent substrate is a hybrid molecule composed of 12 bases of RNA and DNA. In specific embodiments, the RNA component of the synthetic RNA fluorescent substrate contains the 4-base recognition site (AUUC) of MazF from S. aureus, which is flanked on both sides by at least four deoxyribonucleotides (i.e., dNdNdNdN-AUUC-dNdNdNdN; SEQ ID NO:1). However, as the skilled artisan will appreciate, the synthetic hybrid substrate is flexible and can accommodate longer RNA-DNA hybrids, e.g., from 4 to 10 ribonucleotides (including the sequence AUUC) flanked by 4 to 10 deoxyribonucleotides on either side. The synthetic hybrid substrate can be produced by chemical synthesis, recombinant techniques or a combination thereof routinely practiced in the art.

To facilitate detection of cleavage of the synthetic hybrid substrate by the toxin of the TA system, the present construct also features a fluorescent marker on one end of the substrate and a quencher on the other end of the substrate (i.e., fluorescent marker-DNA-RNA-DNA-quencher). Fluorescent marker-quencher pairs are well-known in the art and routinely used in fluorescent hybridization reactions (Marras 2006. Meth. Mol. Biol. 335:3-16). Examples of suitable fluorescent markers of use in the instant construct include, but are not limited to, FAM, TET, HEX, Cy3, TMR, ROX, TEXAS RED, and Cy5 fluorophores. Fluorophores with an emission maximum between 500 and 550 nm, such as FAM, TET and HEX, are best quenched by quenchers with absorption maxima between 450 and 550 nm, such as dabcyl and BHQ-1. Fluorophores with an emission maximum above 550 nm, such as rhodamines (including TMR, ROX and TEXAS RED) and Cy dyes (including Cy3 and Cy5) are best quenched by quenchers with absorption maxima above 550 nm (including BHQ-2). Other quenchers include DDQ-I (absorption maximum of 430 nm), QSY-7 (absorption maximum of 571 nm), DDQ-II (absorption maximum of 630 nm), and QSY-21 (absorption maximum of 660 nm).

The present invention is also a whole cell screening assay for identifying compounds that inhibit growth of the bacterial cells. The assay is based on screening a drug for activity to disrupt a toxin-antitoxin complex in a whole cell bacterial cell assay and comprises contacting a bacterial cell expressing a wild-type toxin-antitoxin complex with a test drug, contacting a bacterial cell that does not express the wild-type toxin-antitoxin complex with the test drug, and determining whether the test drug reduces the growth of the bacterial cell expressing the wild-type toxin-antitoxin complex as compared to the growth of the bacterial cell that does not express the wild-type toxin-antitoxin complex, wherein a reduction in bacterial growth is indicative of activity to disrupt a toxin-antitoxin complex in the bacterial cell. The goal of the assay will be determination of minimum inhibitory concentration (MIC) doses for the drugs. Determining MIC doses is a standard assay in development of antibiotic drugs and methods are well known in the art. For example, MIC can be determined according to the Clinical and Laboratory Standards Institute guidelines. It is contemplated that the assay can be used to identify compounds that inhibit growth of a variety of bacteria including but not limited to S. aureus, E. coli 0157. Listeria, and M. tuberculosis. In a preferred embodiment the bacterial strains tested are wild-type S. aureus versus MazEFsa mutant S. aureus.

Agents, compounds or test drugs which can be screened in accordance with the method of the present invention are generally derived from libraries of agents or compounds. Such libraries can contain either collections of pure agents or collections of agent mixtures. Examples of pure agents include, but are not limited to, proteins, polypeptides, peptides, nucleic acids, oligonucleotides, carbohydrates, lipids, synthetic or semi-synthetic chemicals, and purified natural products. Examples of agent mixtures include, but are not limited to, extracts of prokaryotic or eukaryotic cells and tissues, as well as fermentation broths and cell or tissue culture supernates. Databases of chemical structures are also available from a number of sources including Cambridge Crystallographic Data Centre (Cambridge, UK) and Chemical Abstracts Service (Columbus, Ohio). De novo design programs include Ludi (Biosym Technologies Inc., San Diego, Calif.), Sybyl (Tripos Associates) and Aladdin (Daylight Chemical Information Systems, Irvine, Calif.).

Once the efficacy of a drug or a combination of drugs has been shown based on the use of the in vitro screening methods of the present invention, there are many different in vivo model systems that can be used by one of skill in the art to further demonstrate efficacy against MSSA, MRSA and CA-MRSA and aid in identification of doses that will be both safe and effective in humans. Such animal model systems are well-accepted and used during development of new human pharmaceuticals that will undergo scrutiny by various regulatory bodies worldwide and approved for use in humans. Examples of such model systems include but are not limited to a guinea pig model of S. aureus wound infection (Kernodle, D. S, and A. B. Kaiser. 1994. Antimicrob. Agents Chemother. 38:1325-1330); a rabbit model of S. aureus abscess in rabbits (Fernandez et al. 1999. Antimicrob. Agent Chemother. 43:667-671); a mouse model of S. aureus skin infection (Gisby, J. and J. Bryant. 2000. Antimicrob. Agents Chemother. 44:255-260); a mouse model of deep dermal S. aureus infection (Godin et al. 2005. J. Antimicrob. Chemother. 55:989-994); and a mouse intraperitoneal infection model (Patel et al. 2004. Antimicrob. Agents Chemother. 48:4754-4761). In such models, drugs can be tested against infections where the infection established is from inoculation of the animal with various strains of S. aureus. Demonstration of efficacy in such models is measured in many ways and would include but not be limited to a reduction in mortality rate, a reduction in bacterial cell counts determined by microscopic examination of tissue or blood samples taken from the animals, or even assessment of wound healing in the animals.

In order to demonstrate the utility of disruption of a toxin-antitoxin complex in vitro as a method for screening for drugs to treat infection and thus provide proof of the concept for drug screening, in vivo experiments were performed. A murine model of subcutaneous infections (abscess) was used. Such models are well-established as methods to screen for in vivo activity to affect infection processes (Tamber et al. 2010. Infect. Immun. 78:3637-3646).

In the present experiments, a bacterial strain ALC6094, which is a derivative of S. aureus strain Newman was used. This strain contains a chromosomal copy of the T7 polymerase gene and the plasmid pG164::mazFsa, which is a shuttle plasmid with an IPTG-inducible T7 promoter driving MazFsa expression. Mice were challenged with 2×10⁷ CFU of bacteria via subcutaneous (s.q.) injection and mixed with 1 mM IPTG immediately before injection. After injection, half of the IPTG-treated animals received two saline injections daily while the remaining half were injected with 1 mM IPTG (s.q.) daily for 2 days. All animals were sacrificed on day 4, including untreated control groups receiving no IPTG. Only animals receiving saline injections developed visible abscesses by day 3, while those animals receiving IPTG induction for MazFsa exhibited erythematous injection sites. Mice receiving IPTG induction for MazFsa had smaller abscess sizes and significantly fewer bacterial counts (CFUs) as compared with the “no IPTG” controls. These data demonstrated that activated MazFsa can diminish virulence of S. aureus, consistent with the toxic activity of MazFsa as predicted from in vitro studies. These data provided direct proof for the utility of using the strategy of releasing MazFsa from the innocuous MazEFsa complex to exert its toxic activities. Thus, drugs that disrupt the MazEF complex would be novel compounds for use in treating S. aureus bacterial infections.

Similar experiments can be performed with the same animal model but employing bacterial strains other than S. aureus. Further, use of this animal model as a method of identifying compounds with efficacy as antibiotics is also contemplated where the goal is to identify compounds that induce growth arrest of parental S. aureus cells, but not mazEFsa mutant cells. Given that MazEFₛₐ is highly homologous to that of S. epidermidis, it is highly likely that compounds identified with the present method would also disrupt the MazEFₛₑ of multidrug resistant (MDR) S. epidermidis as well. Further, given that TA systems are common in many pathogenic bacteria, this approach will provide for identification of novel agents against TA systems in other pathogenic and biodefense-related bacteria, that would include but not be limited to E. coli 0157, Listeria, and M. tuberculosis).

As a result, the present invention is a method of screening a drug for efficacy as an antibiotic which comprises inoculating a first mouse with a bacterial strain expressing a wild-type toxin-antitoxin complex, inoculating a second mouse with a bacterial strain that does not express the wild-type toxin-antitoxin complex, administering to both the first mouse and the second mouse a test drug and measuring a volume of bacterial abscess that forms subcutaneously on said first mouse and said second mouse, wherein a reduction in said bacterial abscess volume or the number of bacteria in the first mouse when compared with abscess volume or number of bacteria in the second mouse is indicative of efficacy of the test drug as an antibiotic compound. In preferred embodiments the bacterial strain is selected from the group consisting of S. aureus, E. coli 0157. Listeria, and M. tuberculosis, and the bacterial strains tested are wild-type S. aureus versus MazEFsa mutant S. aureus.

Other screening models for in vivo drug efficacy can also be employed. For example, the efficacy of a drug that has been screened by the methods of the present invention and shown to have activity to inhibit growth of S. aureus including methicillin-susceptible S. aureus (MSSA), methicillin-resistant S. aureus (MRSA) and community-acquired methicillin-resistant S. aureus (CA-MRSA) can be further examined using the model described by Patel et al. (2004. Antimicrob. Agents Chemother. 48:4754-4761). Briefly, Swiss mice (6 mice per dose group, 4 weeks of age) will be inoculated intraperitoneally (i.p.) with 0.5 ml of bacterial suspension so that each mouse will receive from 2×10⁸ to 3×10⁸ CFU of isolate. The drug to be tested, or the combination of drugs to be tested, is then at a dose shown to be effective in vitro but also known to be safe in animals. The doses to be tested are routinely chosen by those of skill in the art by using clinical judgment based on results of in vitro pharmacological assays. For example, doses can be ones that are equivalent to an ED₁₀, an ED₂₅, an ED₅₀, and an ED₇₅ for inhibiting bacterial growth in vitro. The drug will be administered at 1 and 4 hours after i.p. inoculation of mice with isolates. The drug to be tested can be administered subcutaneously, intravenously, or orally. A vehicle control group will be used. All mice are observed for survival up to 7 days. Efficacy of the test drug will be measured as an increased survival rate as compared to control animals (untreated) and as compared to survival in a group of animals administered a positive control agent (e.g., an antibiotic known to have efficacy to treat S. aureus).

Using any in vivo method for drug screening of the present invention, the candidate drugs to be tested for antibiotic activity can be given orally, by intravenous injection or dermally. If dermal administration is to be tested, the drug will be spread over the area of the abscess. The dose of test drug to be administered will be chosen based on the results of in vitro studies of inhibition of bacterial growth. As discussed above, doses can be chosen based on the percentage of growth inhibition seen in vitro.

It is contemplated that one of skill in the art will choose the most appropriate bacterial strains for testing in vivo depending on the type of drug product being developed.

Once a test drug or a combination of drugs has shown to be effective in vivo, the frequency in the emergence of resistance in vivo can be assessed. If the rate of emergence of resistance is low, clinical studies can be designed based on the doses shown to be safe and effective in animals. One of skill in the art will design such clinical studies using standard protocols as described in textbooks such as Spilker (2000. Guide to Clinical Trials. Lippincott Williams & Wilkins: Philadelphia).

The following non-limiting examples are provided to further illustrate the present invention.

### EXAMPLES

### Example 1

### Library Screen

To identify compounds as antibiotic compounds with potential activity against S. aureus, experiments were performed wherein MazEF complex of S. aureus was incubated with the hybrid molecule substrate and the compounds, which were to be tested for activity to disrupt the MazEF complex. Compounds that disrupted the complex cleaved the hybrid fluorescence substrate and yielded detectable fluorescence. The results of this screening assay identified the compounds listed in Table 1.

TABLE 1

| | | ChemBridge | MIC |
|---|---|---|---|
| Compound # | Compound Structure and Name | ID # | (μg/ml) |
| 1 | 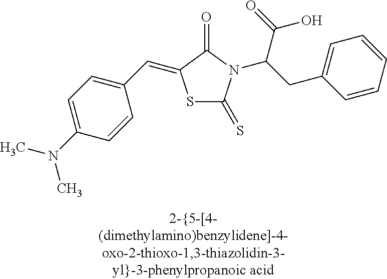 | 6048022 | 12.5 |
| 2 | 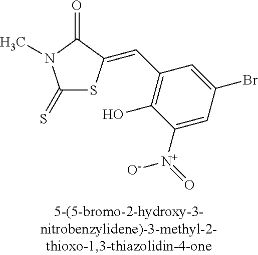 | 5902920 | 12.5 |
| 3 | 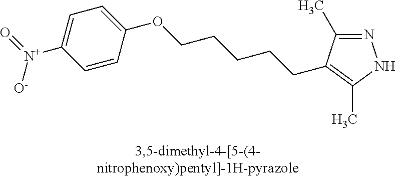 | 5362508 | ND |
| 4 | 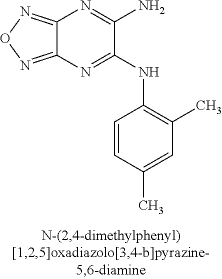 | 5380590 | 12.5 |
| 5 | 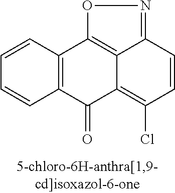 | 5468117 | 6.25 |
| 6 | 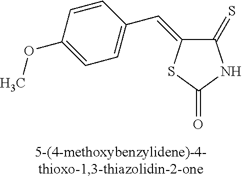 | 5761926 | 0.78 |
| 7 | 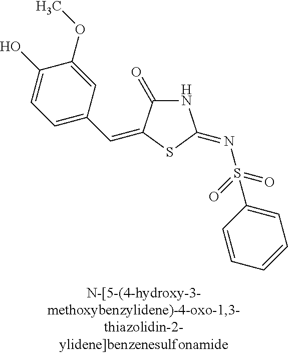 | 5957303 | 100 |
| 8 | 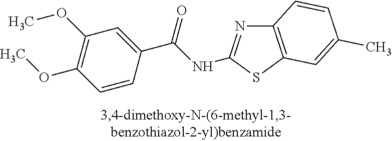 | 5564414 | ND |
| 9 | 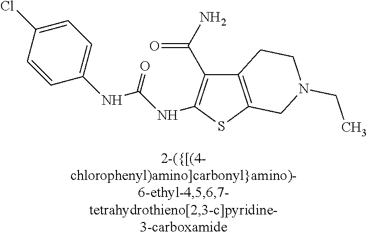 | 6131237 | 100 |
| 10 | 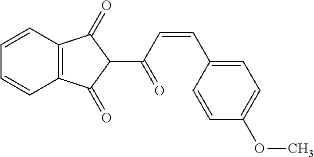 | 5215283 | 100 |

Once the compounds of the present invention were identified through the MazEF screening assay described above, the activity of the compounds to inhibit growth of S. aureus in vitro was tested using the standard method of determining Minimum Inhibitory Concentrations (MICs). Such methods are well known to those of skill in the art since determining MIC doses is a standard assay in development of antibiotic drugs. For example, MIC values can be determined according to the Clinical and Laboratory Standards Institute guidelines. In Table 1, the MIC values are presented for each of the ten compounds identified through the MazEF screening assay as having potential for antibiotic activity in S. aureus.

In addition to the above screen, an additional screen was employed. In this second assay, compounds from a compound library (natural or synthetic compounds) were incubated with either wild-type bacteria (with the MazEF complex) or a mazEF mutant of S. aureus. When the compound of interest disrupts the MazEF complex of S. aureus to free up the toxin (MazF), the mRNA-cleavage toxicity of the toxin is available to kill the parental strain; however, the isogenic mazEF mutant will not be killed. Using this assay, the compounds listed in Table 2 were identified as inhibiting the growth of wild-type strain Newman but not the isogneic mazEF mutants.

TABLE 2

| Compound # | Structure | ChemBridge ID # |
|---|---|---|
| 11 | 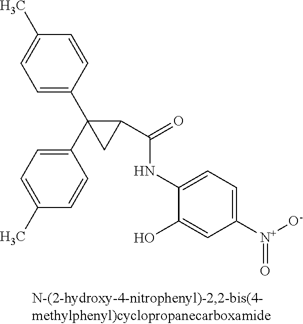 | 5634518 |
| 12 | 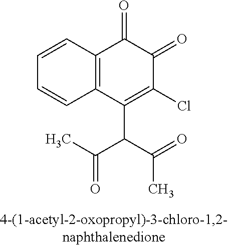 | 5286499 |
| 13 | 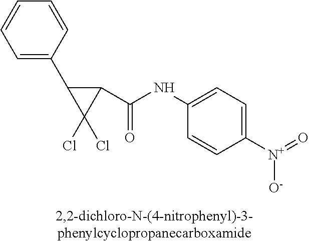 | 6079510 |
| 14 | 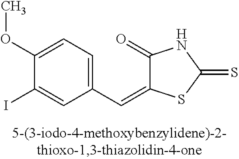 | 5486272 |
| 15 | 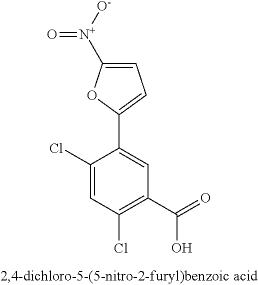 | 6047950 |
| 16 | 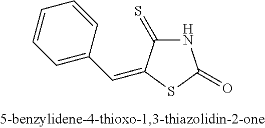 | 5765232 |

These results demonstrate the effectiveness of the using the instant assay in identifying compounds of use in inhibiting the growth of S. aureus and in the prevention and/or treatment of an S. aureus infection.

### Example 2

### In Vivo Model for Drug Efficacy

Balb/C female mice (20-22 grams) were used to assess the utility of disrupting MazEFsa activity in vivo as a potential method for testing for drug efficacy. The mice were infected subcutaneously with 200 μl of 1.5×10⁸ CFU/ml of bacteria. The bacterial strain used was ALC6094, which is a derivative of S. aureus strain Newman containing a chromosomal copy of the T7 polymerase gene and the plasmid pG164::mazFsa, which is a shuttle plasmid with an IPTG-inducible T7 promoter driving MazFsa expression. Control mice were infected with the bacteria but received no further injections. Two treatment groups were tested: 1) mice injected with ALC6094 that was mixed with IPTG immediately before injection (1 ml of a 5 mM solution of IPTG); and 2) mice injected with ALC6094 mixed with IPTG and then given daily IPTG injections for two subsequent days. Animal were sacrificed on day 4 after injection. Only mice receiving saline control developed visible abscesses by day 3, while those animals receiving IPTG had visible erythematous injection sites. Mice receiving IPTG induction for MazFsa had smaller abscess sizes and significantly fewer bacterial counts (CFUs) as compared with the “no IPTG” controls (see Table 3 below). These data demonstrated that activated MazFsa can diminish virulence of S. aureus, consistent with the toxic activity of MazFsa as predicted from in vitro studies.

TABLE 3

| | Log₁₀ CFU/ | P values vs. |
|---|---|---|
| Group (# mice) | abscess | control |
| Untreated (n = 7) | 8.02 ± 0.34 | |
| ALC6094 plus IPTG | 6.57 ± 0.83 | 0.000001 |
| mixture (n = 9) | | |
| ALC6094 plus IPTG | 6.59 ± 0.95 | 0.000006 |
| mixture plus IPTG | | |
| daily injection | | |
| (n = 9) | | |

What is claimed is:

1. A method of screening a drug for activity to disrupt a toxin-antitoxin (TA) complex in a bacterial cell comprising contacting a TA complex with a test drug and determining whether the test drug increases cleavage of mRNA by the toxin, wherein an increase in cleavage of mRNA by the toxin is indicative of activity to disrupt a TA complex in the bacterial cell.

2. The method of claim 1, wherein the TA complex is a MazEF complex.

3. The method of claim 2, wherein the MazEF complex is from a S. aureus cell.

4. The method of claim 1, wherein the cleavage of the mRNA by the toxin is determined by measuring cleavage of a synthetic RNA substrate.

5. The method of claim 4, wherein the synthetic RNA substrate comprises a MazF recognition site (AUUC).

6. The method of claim 4, wherein the RNA of the substrate is flanked on each end by at least four deoxyribonucleotides.

7. The method of claim 4, wherein the synthetic RNA substrate comprises a fluorescent marker-quencher pair.

8. The method of claim 1, wherein cleavage of the RNA by the toxin is determined by measuring cleavage of a synthetic RNA-DNA substrate comprising the 4-base MazF recognition site (AUUC) flanked on each side by at least four deoxyribonucleotides, wherein said substrate comprises a fluorescence marker at one end and a quencher at the other end.

9. A synthetic substrate comprising a MazF recognition site (AUUC) flanked on either side by at least four deoxyribonucleotides, wherein said substrate comprises a fluorescence marker at one end and a quencher at the other end.

10. A method of screening a drug for activity to disrupt a toxin-antitoxin complex in a whole cell bacterial cell assay comprising contacting a bacterial cell expressing a wild-type toxin-antitoxin complex with a test drug, contacting a bacterial which does not express the toxin-antitoxin complex with the test drug, and determining whether the test drug reduces the growth of the bacterial cell expressing the wild-type toxin-antitoxin complex as compared to the growth of the bacterial cell which does not express the toxin-antitoxin complex, wherein a reduction in bacterial growth is indicative of activity to disrupt a toxin-antitoxin complex in the wild-type bacterial cell.

11. The method of claim 10 wherein the bacterial strain is selected from the group consisting of S. aureus, E. coli 0157. Listeria, and M. tuberculosis.

12. The method of claim 10 wherein the bacterial strains tested are wild-type S. aureus and MazEFsa mutant S. aureus.

13. A method of screening a drug for efficacy as an antibiotic comprising inoculating a first mouse with a bacterial strain expressing a wild-type toxin-antitoxin complex, inoculating a second mouse with a bacterial strain which does not express the toxin-antitoxin complex, administering to both the first mouse and the second mouse a test drug and measuring a volume of bacterial abscess that forms subcutaneously on said first mouse and said second mouse, wherein a reduction in said bacterial abscess volume or number of bacteria in the first mouse when compared with abscess volume or number of bacteria in the second mouse is indicative of efficacy of the test drug as an antibiotic compound.

14. The method of claim 13 wherein said bacterial strain is selected from the group consisting of S. aureus, E. coli 0157. Listeria, and M. tuberculosis.

15. The method of claim 13 wherein said bacterial strain is wild-type S. aureus and mazEFsa mutant S. aureus.


SEQUENCE LISTING (1 sequence)

SEQ ID NO: 1 - DNA, 12 bp, Artificial sequence
  Synthetic oligonucleotide
nnnnauucnnnn